(19) United States
(12) Patent Application Publication
(10) Pub. No.: US 2026/0132363 A1
(43) Pub. Date: May 14, 2026

(54) A CULTURE DEVICE

(71) Applicant: Oribiotech LTD, London, Greater London (GB)

(72) Inventor: Farlan Singh Veraitch, London, Greater London (GB)

(21) Appl. No.: 19/119,063

(22) PCT Filed: Oct. 6, 2023
(86) PCT No.: PCT/GB2023/052593

§ 371 Date: Apr. 7, 2025

(30) Foreign Application Priority Data
Oct. 7, 2022 (GB) 2214725.0

(51) Int. Cl. C12M 1/00; C12M 1/26; C12M 3/06

(52) U.S. Cl. CPC C12M 23/26; C12M 23/02; C12M 23/20; C12M 27/16; C12M 33/00

(57) ABSTRACT

There is provided a culture device having a base with a first surface and a second surface fixedly arranged with respect to the first surface, and a flexible side wall extending from the base and defining an internal volume of the device, the first surface and the second surface being non-coplanar.

### CROSS-REFERENCE TO RELATED APPLICATIONS

This application is a national phase entry under 35 U.S.C. § 371 of International Patent Application PCT/GB 2023/052593, filed Oct. 6, 2023, designating the United States of America and published as International Patent Publication WO 2024/074838 A1 on Apr. 11, 2024, which claims the benefit under Article 8 of the Patent Cooperation Treaty of British Patent Application Serial No. 2214725.0, filed Oct. 7, 2022.

### TECHNICAL FIELD

The present disclosure relates to a device, particularly a culture device for culturing cells, a method of culturing cells, and a system for culturing cells.

### BACKGROUND

It is generally known in the field of cell culture that cellular growth proceeds from a so-called initial “lag phase,” in which cells adapt to the culture environment and adopt a slow growth rate, to a so-called “log phase,” in which cells proliferate exponentially and consume nutrients in their growth medium. As the cells consume the nutrients in their growth medium, it becomes necessary to remove the spent medium and introduce fresh growth medium in order to ensure that the cells continue to proliferate. Furthermore, as the density of the cell culture increases as cells proliferate, it becomes necessary to transfer the cell culture from one culture device to another, larger, culture device, having fresh growth medium therein. This process is known as passaging of the cell culture. This is also required for adherent-type cell cultures, in which cells adhere, or attach, to an appropriate surface. In adherent-type cell cultures, the adherent-type cells will proliferate and eventually occupy the entirety of the surface area available within a culture device. Thus, it becomes necessary to transfer the adherent-type cell culture from one surface to another, larger, surface in order to maintain a desired proliferation rate.

Generally, the passaging process involves transferring cells from one culture container to another, often larger, container. This process is further complicated for adherent-type cells, in which cells that are adhered, or attached, to a surface must be dissociated, or detached, from such a surface prior to transferring to another culture container. There are many dissociation procedures known in the art, such as shaking or agitating the culture vessel, vigorous pipetting, scraping of the cells from the surface, or enzymatic dissociation using reagents such as Trypsin, Dispase, TrypLE™ (Thermo Fisher Scientific), collagenase, ethylenediaminetetraacetic acid (EDTA), or Accutase™ (Innovative Cell Technologies).

The current process of transferring cells between various culture containers has many disadvantages. Firstly, there is a risk that the cells are introduced into, or through, a non-sterile environment, through the use of interconnecting tubing, additional containers or other devices that may not be adequately sterilized prior to use. Secondly, the process of transferring cells requires manual intervention, especially in the context of adherent-type cell cultures, which hence introduces the risk of operator error, and potentially compromising sterility, during use. Thirdly, cell yields are often detrimentally affected by such a transferring process, since it is difficult to ensure that most, or all, of the cells have been removed from the first container, and then transferred to the second container. In particular, cells are often lost through pipette ends, tubing or the like during the transfer process. Fourthly, in currently known culture containers, it is often cumbersome to remove the finalized cellular product from the container at the end of the process. Fifthly, the use of several containers is not an efficient use of space, thereby affecting scalability of a process. Often, cells are removed by pipetting, or through pumping mechanisms, which can introduce an extra layer of complexity to the system, having an associated risk of compromising sterility and being at risk of operator error. Additionally, such pipetting for the transfer of materials necessitates a laminar flow hood, which requires the highest grade of personal protective equipment, as well as a particulate free environment. In this way, typical transfer of material through pipetting results in higher costs, a more time consuming and labor consuming process, and requires highly skilled operators.

Therefore, it is an object of the present disclosure to solve, or at least mitigate, the abovementioned drawbacks. In particular, it is an object of the present disclosure to provide a culture device and method that avoids the need for cells to be transferred-or “passaged”-between two or more culture devices, for example, two, three, four or more culture devices. More particularly, it is an object of the present disclosure to provide a culture device and method in which cells can be passaged, or otherwise moved, within a single culture device, thereby ensuring an aseptic environment and higher cell yields. Such a culture device and method may also be more suited to automation. It is also an object of the present disclosure to provide a culture device in which material, for example, cells, is more easily removed. It is yet a further object of the present disclosure to provide a culture device in which material, for example, cells, can be mixed, resuspended in medium, or otherwise agitated.

### BRIEF SUMMARY

According to one aspect of the present disclosure, there is provided a device comprising a base comprising a first surface and a second surface fixedly arranged with respect to the first surface, and a side wall extending from the base and defining an internal volume of the device, wherein the first surface and the second surface are non-coplanar.

That is, the present disclosure provides a device, for example, a culture device or a device that may be suitable for culturing material, such as cellular material. The device includes a base and a side wall upstanding from the base. The base includes more than one surface, i.e., a plurality of surfaces. At least one of those surfaces forms an angle with respect to a horizontal, i.e., a transverse, plane, extending perpendicularly to the central longitudinal axis of the device, particularly at a predetermined, pre-set, or fixed, angle. That is, the device may comprise a plurality of surfaces, each surface being fixedly arranged (i.e., not capable of movement) with respect to one another. The transverse plane may be defined at least partially by the base, such as by a particular point of the base disposed within the transverse plane, or by one side of the base that extends within the transverse plane, as described further below.

Put another way, without reference to a particular plane, e.g., a horizontal or transverse plane, the base is non-planar. That is, the base includes a plurality of surfaces, at least one of the surfaces being non-coplanar with respect to the other surface or surfaces. That is, at least one of the surfaces, such as a first surface, may form an angle of between 1 degree and 179 degrees with respect to another surface, such as a second surface. The angle may be positive or negative with respect to the transverse plane. That is, one culture surface may form an angle with respect to another culture surface that is from +1 degree to +179 degrees, or from-1 degree to-179 degrees. In particular examples, one surface may form an angle with respect to another surface that is from 90 degrees to 179 degrees.

Put yet another way, the base includes a plurality of surfaces, such as a first surface and a second surface, each surface extending within a plane. The planes of the first surface and the second surface are non-coplanar. That is, the planes of the first surface and the second surface form an angle. The angle may be that as described above or that as described in more detail below.

In some examples, there is provided a culture device comprising a base and a side wall extending from the base in an axial direction along a central longitudinal axis, the side wall defining an internal volume of the device, wherein the base comprises a plurality of surfaces, wherein at least one of the surfaces is non-coplanar with respect to the other surface or surfaces.

Preferably, the side wall is a flexible side wall. Optionally, the flexible side wall is a compressible side wall. Alternatively, the side wall is a rigid side wall.

This provides the advantage that each surface can, independently, be arranged into a horizontal position, i.e., within the horizontal or transverse plane. In this way, as the device is reorientated from one surface being horizontal to another surface being horizontal, material such as a cell culture can be transferred, or “passaged,” between the surfaces without having to leave the device. Accordingly, the requirement of multiple devices is avoided. In this way, the device ensures a continuous aseptic environment, reduces operator handling and thus associated errors, and is more suited to automated processes. Furthermore, this provides the advantage of enabling low volume cell cultures, such as suspension-type cell cultures, to be mixed, agitated or resuspended prior to addition of further cell culture medium into the container. For example, volumes of approximately 10 mL to approximately 200 mL of settled suspension-type cells may be resuspended within their medium, prior to addition of further medium into the container, often referred to as “expansion” of the volume.

It is noted that any appropriate material may be cultured within the culture device described herein, for example, but by no means any limitation, biological material, bacteria, algae, synthetic meat, human or animal tissue, or human or animal cells. Particularly, the culture device described herein is suitable for culturing adherent-type cells and/or suspension-type cells.

Each of the plurality of surfaces may be fixedly arranged with respect to one another. That is, each one of the plurality of surfaces is provided in a fixed manner with respect to one another. In this respect, the surfaces are statically arranged, i.e., cannot be moved relative to one another. This may be achieved by virtue of rigid surfaces and/or a rigid base.

Optionally, in the case of a substantially circular base, each surface is formed as an angled, or sloped, sector. That is, they may each be formed as a sloped wedge. Optionally, in the case of a substantially circular base, each surface is formed as an angled, or sloped, cylindrical sector. Optionally, each sector may slope downwardly toward a point or axis of the base, such as a central origin or axis thereof.

Optionally, the plurality of surfaces comprises a first surface and a second surface. Optionally, the second surface is separated from the first surface. The first surface and the second surface may have any appropriate arrangement, such as a side-by-side, or adjacent, arrangement, or a concentric arrangement.

Optionally, the first surface, or a plane in which the first culture surface is disposed or located, forms a first angle with respect to the horizontal, i.e., a transverse plane extending substantially perpendicularly to a central longitudinal axis of the device.

Optionally, the first angle is from approximately 1 degree to approximately 179 degrees.

Optionally, the first angle is from approximately 1 degree to approximately 90 degrees.

Optionally, the first angle is from approximately 1 degree to approximately 45 degrees.

Optionally, the first angle is from approximately 1 degree to approximately 30 degrees.

Optionally, the first angle is from approximately 5 degrees to approximately 30 degrees.

Optionally, the first angle is from approximately 5 degrees to approximately 25 degrees.

Optionally, the second surface, or a plane in which the second surface is disposed or located, forms a second angle with respect to the horizontal, i.e., a transverse plane extending substantially perpendicularly to a central longitudinal axis of the device.

Optionally, the second angle is from approximately 1 degree to approximately 179 degrees.

Optionally, the second angle is from approximately I degree to approximately 90 degrees.

Optionally, the second angle is from approximately 1 degree to approximately 45 degrees.

Optionally, the second angle is from approximately 1 degree to approximately 30 degrees.

Optionally, the second angle is from approximately 5 degrees to approximately 30 degrees.

Optionally, the second angle is from approximately 5 degrees to approximately 25 degrees.

Optionally, the first angle and the second angle are different, or non-equal.

Optionally, each of the first angle is approximately from 1 degree to approximately 45 degrees, optionally from approximately 5 degrees to approximately 25 degrees, wherein each of the first angle and the second angle is different, or non-equal. Any of the above contemplated ranges may be utilized in combination.

Optionally, a surface area of the first surface and a surface area of the second surface are equal. Alternatively, the surface area of the first surface and the surface area of the second surface are non-equal.

Optionally, a ratio between a surface area of the first surface and a surface area of the second surface is from approximately 1:9 to approximately 1:1.

Optionally, the ratio is approximately 1:9, or approximately 1:8, or approximately 1:7, or approximately 1:6, or approximately 1:5, or approximately 1:4, or approximately 1:3, or approximately 1:2 or approximately 1:1.

It will be appreciated that such a ratio may represent the surface area of the first surface with respect to the second surface, or the second surface to the first surface.

Optionally, the device and/or base (i.e., the device, the base, or the device and the base) is moveable about an axis to enable each of the first and second surfaces to be positioned in a horizontal plane. This may be the case where the first, the second or both the first and second surfaces form an angle with respect to the horizontal. The axis may be coaxial with the adjoining region between the first and second surfaces. In some examples, the axis may be formed non-coaxially with the adjoining region between the first and second surfaces.

Optionally, the base further comprises a third surface fixedly arranged with respect to, and non-coplanar with, each of the first surface and the second surface. That is, the third surface may be fixedly arranged with respect to the first surface and the second surface. That is, the third surface may be non-coplanar with the first surface and the second surface.

Optionally, the device and/or base (i.e., the device, the base, or the device and the base) is moveable about two axes to enable each of the first, second and third surfaces to be positioned in a horizontal plane. That is, the base may be moveable about a first axis to enable one or more of the surfaces to be positioned in a horizontal plane. That is, the base may be moveable about a second axis to enable one or more of the surfaces to be positioned in a horizontal plane. In some examples, the axes may be coaxial with the adjoining region between surfaces. In some examples, the axes may be formed perpendicularly to one another. Optionally, the base is moveable about more than two axes to enable each of the first, second and third surfaces to be positioned in a horizontal plane. Any number of axes may be contemplated.

Optionally, the third surface is separated from the first surface and the second surface. The first surface, the second surface and the third surface may have any appropriate arrangement, such as a side-by-side, or adjacent, arrangement, a concentric arrangement, or a triangular, i.e., circular sector or cylindrical sector, arrangement.

Optionally, the third surface, or a plane in which the third surface is disposed or located, forms a third angle with respect to the horizontal, i.e., a transverse plane extending substantially perpendicularly to a central longitudinal axis of the device.

Optionally, the third angle is from approximately 1 degree to approximately 179 degrees.

Optionally, the third angle is from approximately 1 degree to approximately 90 degrees.

Optionally, the third angle is from approximately 1 degree to approximately 45 degrees.

Optionally, the third angle is from approximately 1 degree to approximately 30 degrees.

Optionally, the third angle is from approximately 5 degrees to approximately 30 degrees.

Optionally, the third angle is from approximately 5 degrees to approximately 25 degrees.

Optionally, the first angle, the second angle and the third angle are different, or non-equal.

Optionally, each of the first angle, the second angle and the third angle is selected from approximately 1 degree to approximately 45 degrees, optionally from approximately 1 degree to approximately 30 degrees, optionally from approximately 5 degrees to approximately 25 degrees, wherein each of the first angle, the second angle and the third angle are different, or non-equal. Any of the above contemplated ranges may be utilized in combination.

Optionally, the first angle is approximately 20 degrees, the second angle is approximately 30 degrees and the third angle is approximately 45 degrees.

Whilst three surfaces have been described above, it will be readily appreciated that further surfaces, such as fourth, fifth, sixth etc., surfaces, may be formed as part of the device. Optionally, each surface is formed at a different angle with respect to the horizontal, i.e., a transverse plane extending substantially perpendicularly to a central longitudinal axis of the device. Optionally, in the event of any number of surfaces, the device and/or base (i.e., the device, the base, or the device and the base) may be moveable about two (or more) axes to enable each of the surfaces to be positioned in a horizontal plane. That is, the base may be moveable about a first axis to enable one or more of the surfaces to be positioned in a horizontal plane. That is, the base may be moveable about a second axis to enable one or more of the surfaces to be positioned in a horizontal plane. In some examples, the axes may be coaxial with the adjoining region between surfaces. That is, the number of axes may be equal to the number of adjoining regions between surfaces. In some examples, the axes may be formed perpendicularly to one another. Optionally, the base is moveable about more than two axes to enable each of the surfaces to be positioned in a horizontal plane. Any number of axes may be contemplated.

Optionally, a surface area of the first surface, a surface area of the second surface, and a surface area of a third surface are equal. Alternatively, the surface area of the first surface, the surface area of the second surface and the surface area of the third surface are non-equal.

Optionally, a ratio between a surface area of the first surface, the second surface and the third surface is approximately 1:2:3.

Optionally, a ratio between a surface area of the first surface, the second surface and the third surface is approximately 1:3:6.

Optionally, the surface area of the first surface is 25 cm², the surface area of the second surface is 75 cm², and the surface area of the third surface is 150 cm².

This provides the advantage that cells may be transferred to increasingly larger surface areas as they proliferate.

Optionally, the base further comprises at least one port. Optionally, the base further comprises at least one outlet.

This provides the advantage that the material, such as cells, may be removed from the device.

Optionally, one or each surface comprises a port, or an outlet. The port or outlet may be provided on the first surface, the second surface and/or the third surface. The port or outlet may be provided on any surface.

Optionally, one or more ports is or are disposed at or adjacent an adjoining region between adjacent surfaces. Optionally, one or more outlets is or are disposed at or adjacent an adjoining region between adjacent surfaces.

This provides the advantage that the maximum amount of material is harvested from the device. In particular, the first surface and/or the second surface may be pivoted so as to direct the material toward the port, thereby ensuring maximum harvesting efficiency.

Optionally, the flexible side wall comprises a corrugated wall.

Optionally, the flexible side wall comprises a bellows side wall. The bellows side wall may include a series of deformable regions interleaved with rigid sections. The flexible side wall, such as a bellows side wall, may be held within an external supporting, optionally surrounding, frame.

This provides the advantage that the compressible wall element is moveable in response to the pivoting of the base, i.e., the first surface and the second surface. This also provides the advantage of providing a compressible device that can be compressed and/or extend by external forces to encourage mixing of the contents.

Optionally, at least one of the surfaces comprises an adherent cell culture substrate or coating.

This provides the advantage that the device is suitable for adherent-type cells, which can thus be “passaged” between the surfaces within the device.

Optionally, the adherent cell culture substrate or coating comprises an extracellular matrix.

Optionally, the adherent cell culture substrate or coating is selected from the group comprising collagen, fibronectin, vitronectin, laminin, gelatine, poly-lysine, cellulose, or a combination thereof.

Optionally, the base comprises polystyrene, polycarbonate, low-density polyethylene, high-density polyethylene, silicone, or a thermoplastic elastomer. In particular embodiments, the base comprises silicone, particularly a substantially gas-permeable silicone.

Optionally, at least one of the surfaces, or each surface, comprises polystyrene, polycarbonate, low-density polyethylene, high-density polyethylene, silicone, or a thermoplastic elastomer. In particular embodiments, the base comprises silicone, particularly a substantially gas-permeable silicone.

Optionally, the base comprises a composite, such as a silicone inner wall and a substantially gas-impermeable outer wall.

Optionally, the base comprises a substantially gas-impermeable thermoplastic elastomer.

The above materials provide multiple advantages. For example, the base or the surface(s) being comprised of polystyrene allows for adherent-type cells to be directly adhered to the surface(s), thereby mitigating the need for an additional substrate or coating. Moreover, the base being comprised of silicone provides gas-permeability at the base of the culture device, thereby avoiding hypoxic conditions during use of the culture device. Yet further, the base being comprised of a thermoplastic elastomer provides the necessary biocompatibility and mechanical integrity for the base of the device, but may provide a gas-impermeable base, which may be advantageous to particular materials to be cultured within the device.

Optionally, the base is rigid. That is, the base comprises a rigid, or non-flexible, material.

Optionally, each of the surfaces is rigid, or non-flexible. That is, each surface comprises a rigid, or non-flexible, material. Each surface may be rigidly disposed, i.e., not able to flex or bend, with respect to one another.

Optionally, the base comprises a first side and a second, opposing, side. The first side may comprise each culture surface. The second side may be substantially planar. The second side may comprise a block of material, such as a wedge, forming the angled nature of one or more surfaces. The second side may comprise a substantially horizontal surface having the block of material extending axially therefrom. The block of material may be integrally formed with the second side. Optionally, the second side comprises a base and at least one wall upstanding from the base in an axial direction, i.e., along a longitudinal axis. The at least one wall may form the angled surface(s).

Optionally, at least one of the surfaces forms an angle with respect to the second side of the base. That is, one or more of the surfaces on the first side of the base forms an angle with respect to the second side of the base. Optionally, the second side of the base defines, i.e., is disposed within, the transverse plane.

Optionally, the device comprises a top, spaced apart from the base by the side wall. As such, the top is disposed at a distal end of the side wall, and the base is disposed at a proximal end of the side wall. The top may include an opening. The opening may be threaded either internally or externally.

Preferably, the device is a culture device. Optionally, the culture device is a biological culture device, such as a cell culture device. In the case of a culture device, the first surface may be a first culture surface and/or the second surface may be a second culture surface. Additional surfaces, e.g., the third surface, may also form culture surfaces, e.g., a third culture surface, etc.

In accordance with another aspect of the disclosure, there is provided a culture device comprising a base and a flexible side wall extending from the base in an axial direction along a central longitudinal axis, the flexible side wall defining an internal volume of the device, wherein the base comprises a plurality of culture surfaces, at least one of the culture surfaces forming an angle with respect to a transverse plane, defined by at least a part of the base, extending substantially perpendicularly to the central longitudinal axis.

In accordance with another aspect of the present disclosure, there is provided a system for culturing cells comprising:
a culture device as described herein; and
an interfacing member operably coupled to a top of the device.

Optionally, the interfacing member is threadingly coupled to a threaded opening of the device, optionally formed in the top wall thereof.

Optionally, the interfacing member comprises one or more ports for the ingress and/or egress of material to/from the device. Optionally, the one or more ports comprise septum seals.

In accordance with another aspect of the present disclosure, there is provided a system for culturing cells comprising:
a device as described herein; and
an actuator configured to engage at least a portion of the device and move the device between a first configuration, in which the first surface is horizontal, and a second configuration, in which the second surface is horizontal.

That is, “horizontal” refers to the horizontal plane, i.e., a transverse plane extending substantially perpendicularly to a central longitudinal axis of the device.

Optionally, the actuator is configured to move the device and/or the base (i.e., the device, the base, or the device and the base) about at least one axis, or two axes, or two or more axes.

Optionally, the actuator is configured to move the device and/or the base (i.e., the device, the base, or the device and the base) about two axes to enable each of the surfaces to be positioned in a horizontal plane. That is, the base may be moveable about a first axis to enable one or more of the surfaces to be positioned in a horizontal plane. That is, the base may be moveable about a second axis to enable one or more of the surfaces to be positioned in a horizontal plane. In some examples, the axes may be coaxial with the adjoining region between surfaces. In some examples, the axes may be formed perpendicularly to one another. Optionally, the base is moveable about more than two axes to enable each of the surfaces to be positioned in a horizontal plane. Any number of axes may be contemplated.

Optionally, the actuator is configured to engage at least a portion of the base and move the base about at least one axis within the transverse plane. In this way, the actuator causes movement between the first and second configurations.

Optionally, the actuator is configured to engage at least a portion of the base and move the base about two axes to enable each of the surfaces to be positioned in a horizontal plane. That is, the base may be moveable about a first axis to enable one or more of the surfaces to be positioned in a horizontal plane. That is, the base may be moveable about a second axis to enable one or more of the surfaces to be positioned in a horizontal plane. In some examples, the axes may be coaxial with the adjoining region between surfaces. In some examples, the axes may be formed perpendicularly to one another. Optionally, the base is moveable about more than two axes to enable each of the surfaces to be positioned in a horizontal plane. Any number of axes may be contemplated.

This provides the advantage that the orientation of each culture surface can be controlled by the actuator, either through manual or automatic manipulation of the actuator.

Optionally, the actuator is electrically coupled to a processor.

Optionally, the processor is configured to actuate the actuator according to a pre-defined set of parameters.

Optionally, the processor may be electrically coupled to one or more sensors of the culture device.

Optionally, the processor may be configured to receive a signal from the one or more sensors and actuate the actuator in response to the received signal.

Optionally, the actuator is further configured to engage at least a portion of the base and move the base along a longitudinal axis. The longitudinal axis may be the central longitudinal axis of the culture device.

This provides the advantage that the entirety of the base is translated, thereby allowing for the mixing of the contents of the culture device by compressing and/or extending the flexible side wall of the culture device.

Optionally, the system further comprises an interfacing member as discussed in the above aspect.

In accordance with another aspect of the present disclosure, there is provided a kit of parts comprising:
a culture device as described herein; and
an interface plate as described herein, arranged to operably couple to the culture device, and/or an actuator as described herein, configured to engage at least a portion of the base and move the base about at least one axis within the transverse plane.

In accordance with another aspect of the present disclosure, there is provided a method of culturing cells, comprising the steps of:
providing a culture device with a first culture surface and a second culture surface, wherein at least one of the first culture surface and the second culture surface forms an angle with respect to a transverse plane, defined by at least a part of the base, extending substantially perpendicularly to the central longitudinal axis;
orientating the first culture surface into a plane that is substantially parallel to, or co-planar with, the transverse plane;
culturing cells on the first culture surface;
orientating the second culture surface into a plane that is substantially parallel to, or co-planar with, the transverse plane, thereby passaging cells from the first culture surface to the second culture surface; and
culturing cells on the second culture surface.

In accordance with another aspect of the present disclosure, there is provided a method of culturing cells, comprising the steps of:
providing a culture device as described herein;
orientating a first culture surface into a plane that is substantially parallel to, or co-planar with, the transverse plane;
culturing cells on the first culture surface;
orientating a second culture surface into a plane that is substantially parallel to, or co-planar with, the transverse plane, thereby passaging cells from the first culture surface to the second culture surface; and
culturing cells on the second culture surface.

In accordance with another aspect of the present disclosure, there is provided a method of culturing biological material, comprising the steps of:
providing a first surface and a second surface that is fixedly arranged with respect to the first surface, wherein the first surface and the second surface are non-coplanar;
orientating the first surface into a plane that is substantially horizontal;
culturing biological material on the first surface;
orientating the second surface into a plane that is substantially horizontal, thereby transferring biological material from the first surface to the second surface; and
culturing biological material on the second surface.

This provides the advantage that biological material, such as a cell culture, can be transferred, or passaged, between the two surfaces without the need to transfer material to a further container. Accordingly, this method ensures a continuous aseptic environment, reduces operator handling and thus associated errors, and is more suited to automated processes.

Optionally, the step of orientating the first surface into a plane that is substantially horizontal comprises rotating the device and/or the base (i.e., the device, the base, or the device and the base) about a first axis. Optionally, the step of orientating the second surface into a plane that is substantially horizontal comprises rotating the device and/or the base (i.e., the device, the base, or the device and the base) about the first axis, or a second-i.e., different-axis.

Optionally, the biological material comprises tissue or cells.

Optionally, the cells are adherent-type cells and each of the first surface and the second surface comprise an adherent cell culture substrate or coating.

Optionally, the method further comprises a step of introducing media into the device.

Optionally, the method further comprises a step of removing media from the device.

Optionally, the method further comprises a step of genetically modifying cellular material within the device.

Optionally, the method further comprises a step of introducing a virus and/or magnetic beads into the device.

Optionally, the method further comprises a step of introducing growth factors, cytokines or the like into the device.

Optionally, the method further comprises a step of removing biological material, such as the cells, from the device.

In accordance with another aspect of the present disclosure, there is provided a method of culturing adherent-type cells, comprising the steps of:
providing a device as described herein;
orientating a first surface into a plane that is substantially parallel to, or co-planar with, a transverse plane;
culturing adherent-type cells on the first surface;
detaching the adherent-type cells from the first surface;
orientating a second surface into a plane that is substantially parallel to, or co-planar with, the transverse plane, thereby passaging the adherent-type cells from the first surface to the second surface; and
culturing the adherent-type cells on the second surface.

This provides the advantage that an adherent-type cell culture can be transferred, or passaged, between the two culture surfaces without the need to transfer cells to a further container. Accordingly, this method ensures a continuous aseptic environment, reduces operator handling and thus associated errors, and is more suited to automated processes.

Optionally, prior to the step of detaching the adherent-type cells from the first culture surface, there is provided a step of washing the adherent-type cell culture.

Optionally, the step of washing may include introducing a washing solution, such as a buffered saline solution, into the device. The step of washing may also include removing the washing solution from the device.

Optionally, the step of detaching the adherent-type cells from the first surface comprises introducing a dissociation reagent into the device. Optionally, the dissociation reagent comprises Trypsin, Dispase, TrypLE™, collagenase, EDTA, or Accutase™, or the like. Generally, any appropriate dissociation reagent or method for detaching the adherent-type cells may be utilized.

Optionally, any adherent-type cells may be cultured in the device. The adherent-type cells may include one or more of mesenchymal stem cells, induced pluripotent stem (IPS) cells, embryonic stem cells (ESCs), cancer cells lines, human embryonic kidney (HEK) cells, HeLa cells, fibroblasts, or epithelial cells, or the like.

Optionally, the method further comprises:
culturing adherent-type cells on a first substrate disposed on the first surface; and/or
culturing adherent-type cells on a second substrate disposed on the second surface.

Optionally, the method further comprises a step of introducing media into the device. Optionally, the method further comprises a step of removing media from the device.

Optionally, the method further comprises a step of genetically modifying cellular material within the device.

Optionally, the method further comprises a step of introducing a virus and/or magnetic beads into the device.

Optionally, the method further comprises a step of introducing growth factors, cytokines or the like into the device.

Optionally, the method further comprises a step of removing material, such as the cells, from the device. In such a step, the cells may be detached from the respective culture surface prior to removing cells.

Optionally, the method discussed herein are carried out in the device discussed herein.

Generally, the methods discussed herein can be carried out in any appropriate order. Yet further, the methods discussed herein may comprise further steps of passaging cells on to further culture surfaces, for example, third or fourth culture surfaces, as required. In those cases, the method may further comprise the step of orientating the third, fourth, etc., surface into a plane that is substantially horizontal. In some examples, the step of orientating the third, fourth, etc., surface into a plane that is substantially horizontal comprises rotating the device and/or the base about the first axis, or a second—i.e., different to the first-axis. In some examples, the axes may be coaxial with the adjoining region between surfaces. In some examples, the axes may be formed perpendicularly to one another. Any number of axes may be contemplated.

The optional features indicated in respect of the methodologies herein are applicable to each and every method “aspect” described herein.

In accordance with yet another aspect, there is provided a use of the device as described herein, or the system as described herein, in the culture of adherent-type cells.

In accordance with yet another aspect, there is provided a use of the device as described herein, or the system as described herein, in the culture of suspension-type cells.

In accordance with yet another aspect, there is provided a kit of parts comprising a device as described herein, and an interfacing member configured to be operably coupled to a top of the device.

As will be clear to the skilled person, various elements, features, and functions are applicable across all aspects of the invention, whether as described as separate aspects or otherwise.

### BRIEF DESCRIPTION OF THE DRAWINGS

Example embodiments of the disclosure are now described, by way of example only, hereinafter with reference to the accompanying drawings, in which:

FIG. 1 illustrates a side view of a culture device according to the present disclosure;

FIG. 2 illustrates a side view of the culture device of FIG. 1 having an interface plate attached;

FIG. 3 illustrates a side view of another culture device according to the present disclosure;

FIG. 4 illustrates a cross-sectional view of the culture device of FIG. 3 having an interface plate attached;

FIG. 5 illustrates a top view of the culture devices of FIGS. 1 to 4;

FIG. 6 illustrates a side view of another culture device according to the present disclosure having an interface plate attached;

FIG. 7 illustrates a top view of the culture device of FIG. 6;

FIG. 8 illustrates a top view of another culture device according to the present disclosure;

FIG. 9 illustrates a view of the culture device along the section A-A of FIG. 8;

FIG. 10 illustrates a view of the culture device along the section B-B of FIG. 8;

FIG. 11 illustrates a view of the culture device along the section C-C of FIG. 8;

FIGS. 12A and 12B illustrate a top view of other culture devices according to the present disclosure;

FIGS. 13A and 13B illustrates a top view other culture devices according to the present disclosure;

FIGS. 14A-14D illustrate a top view of (FIG. 14A) a square base, (FIG. 14B) another square base, (FIG. 14C) a triangular base, and (FIG. 14D) an octagonal base of various culture devices according to the present disclosure;

FIG. 15 illustrates the culture device of FIGS. 3 and 4 in a first configuration during use; and

FIG. 16 illustrates the culture device of FIGS. 3 and 4 in a second configuration during use.

### DETAILED DESCRIPTION

The described example embodiment relates to a device and a method. They primarily relate to devices and methods in cell processing, particularly cell and/or gene therapy, but are not limited thereto. As will be appreciated by the person skilled in the art, the present device and method are readily applicable to adherent-type cells, suspension-type cells, human-derived cells, animal-derived cells, and the like. The present device and method may be utilized in cell and/or gene therapy, tissue engineering, cellular agriculture, including laboratory or cell grown meat products, water treatment and other like technical fields. Moreover, the present device may be suitable in other fields, such as chemical processing, agriculture and the like. The present device and method is not limited to any one particular use as described herein. The use of ‘culture device’ and ‘culture surface’ are not intended to be limiting in any manner, but instead are mere examples of the device and surfaces that may be utilized.

Certain terminology is used in the following description for convenience only and is not limiting. The words ‘upper,’ ‘lower,’ ‘upwardly,’ and ‘downwardly’ designate directions in the drawings to which reference is made and are with respect to the described component when assembled and mounted. The words ‘inner,’ ‘inwardly,’ ‘outer,’ and ‘outwardly’ refer to directions toward and away from, respectively, a designated centerline or a geometric center of an element being described (e.g., a central axis), the particular meaning being readily apparent from the context of the description. Further, the terms ‘proximal’ (i.e., nearer to) and ‘distal’ (i.e., away from) designate positions relative to a body or a point of attachment.

Further, as used herein, the terms ‘connected,’ ‘affixed,’ and the like are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, ‘first,’ ‘second,’ ‘third,’ etc. merely indicate that different instances of like objects are being referred to and are not intended to imply that the objects so described must be in a given sequence, either temporally, spatially, in ranking or in any other manner. Like reference numerals are used to depict like features throughout, with differing examples raised by an order of 100.

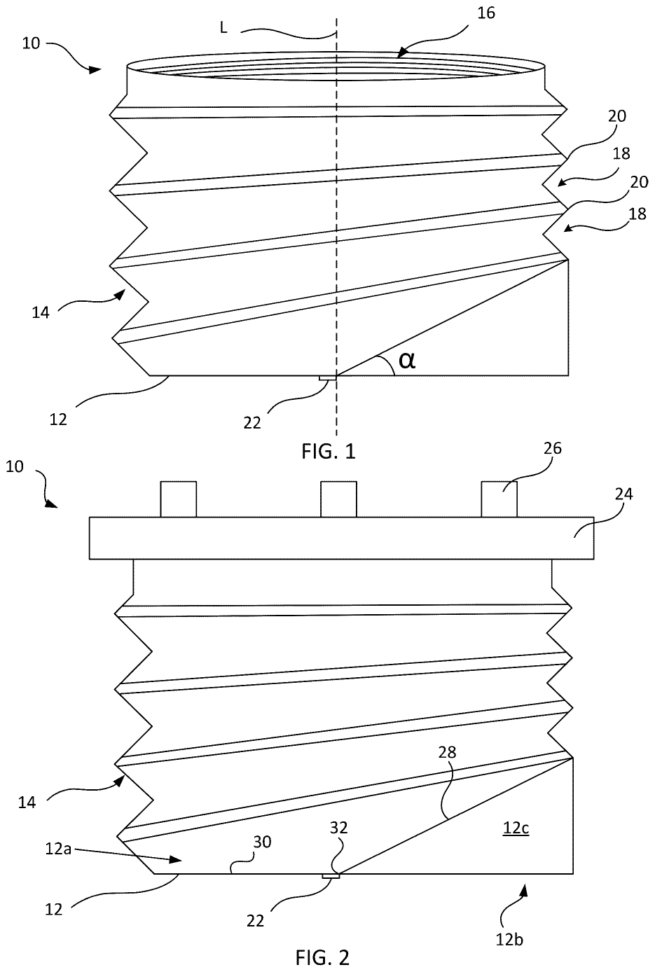

FIGS. 1 and 2 illustrate a culture device 10 including a base 12 and a flexible side wall 14. The flexible side wall 14 extends along a central longitudinal axis L of the culture device, that is in an axial direction, from the base 12. The flexible side wall 14 extends from a proximal end, adjacent and connected to the base 12, toward a distal end. The distal end includes an opening 16, the internal circumference of which may be threaded. The flexible side wall 14 is formed as a bellows side wall and includes a series of deformable regions 18 interleaved with rigid regions 20 therebetween. The culture device 10 also includes an optional outlet port 22, and an interface plate 24 coupled thereto includes a plurality of optional inlet ports 26, as shown in FIG. 2, which are formed as septum seals and enable the ingress and/or egress of material to/from the culture device 10.

Generally, the base 12 comprises a rigid material and the flexible side wall 14 comprises a resilient material. The base 12 may be composed of polystyrene, polypropylene, polycarbonate, low or high density polyethylene (LDPE or HDPE), a thermoplastic elastomer, silicone or the like. The flexible side wall 14 may be composed of silicone, a substantially gas-permeable or substantially gas-impermeable thermoplastic elastomer, a flexible polymeric film, or the like. Any combination of materials for the base 12 and the flexible side wall 14 is contemplated. Likewise, in embodiments not illustrated, the side wall may be composed of a rigid material.

With particular reference to FIG. 2, the culture device 10 includes an interface plate 24 coupled to the opening 16 thereof. The interface plate 24 may be threadingly coupled to the threaded interior of the opening 16. Equally, other couplings, such as a push fit or clip fit, are contemplated. The interface plate 24 comprises a plurality of ports 26, formed as septum seals, for the ingress and/or egress of material through the interface plate 24 and into the culture device 10. The plurality of ports 26 may provide access to the culture device 10 through the use of a needle-based connector (not shown).

As illustrated in FIG. 2, the base 12 of the culture device 10 includes a first side 12a and a second, opposing, side 12b. The first side 12a forms a first culture surface 28 and a second culture surface 30 separated by an adjoining region 32. The first culture surface 28 and the second culture surface 30 are located at a fixed angled with respect to one another as will be described further herein. As illustrated in FIG. 1, the first culture surface 28 is angled with respect to the second side 12b of the base 12, forming a fixed angle a with respect to that second side 12b. In this instance, the second side 12b extends in, and is disposed within, a transverse i.e., horizontal-plane extending perpendicularly to the central longitudinal axis L. That is, the second side 12b is planar. Referring again to FIG. 2 in particular, the angle a between the first culture surface 28 and the second side 12b is fixed and is formed by virtue of a block of material 12c. The block of material 12c is substantially triangular in cross-section. In this way, the second side 12b of the base 12 is provided as a continuous—i.e., flat—surface, and the block of material 12c provides the angled first culture surface 28. Put another way, block of material 12c is provided as a so-called wedge, thereby providing the fixed angled surface 28.

The first culture surface 28 and the second culture surface 30 may be of the same, or a different, material. In some examples, particularly relevant for culturing adherent-cell types, the first culture surface 28 and/or the second culture surface 30 include a coating or substrate to allow for cell attachment, or are composed of appropriate materials to allow for cell attachment, such as polystyrene. The adjoining region 32 generally separates the first culture surface 28 and the second culture surface 30. The culture device 10 includes the plurality an optional outlet port 22 disposed on the second culture surface 30 adjacent to the adjoining region 32, although it may alternatively be formed at the adjoining region 32 or on the first culture surface 28. In the present example, one port 22 is shown, but more, or no, ports are equally contemplated.

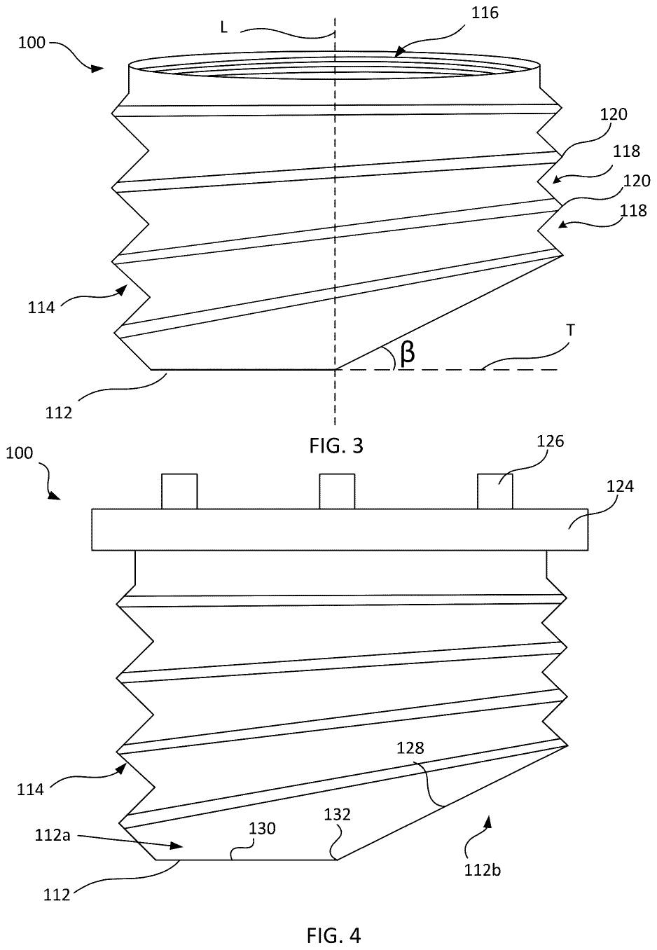

FIGS. 3 and 4 illustrate another culture device 100 that is substantially similar to culture device 10 of FIGS. 1 and 2, respectively, and so like elements will not be described in further detail. In the examples of FIGS. 3 and 4, no outlet port (reference 22 in FIGS. 1 and 2) is provided. Additionally, in the examples of FIGS. 3 and 4, no block of material (reference 12c in FIG. 2) is provided. In this way, the base 112 is provided with a fixed angle ß extending between the first culture surface 128 and a transverse plane T extending perpendicularly to the central longitudinal axis L. The transverse plane T is partially defined by the base 112, particularly by second culture surface 130 and adjoining region 132.

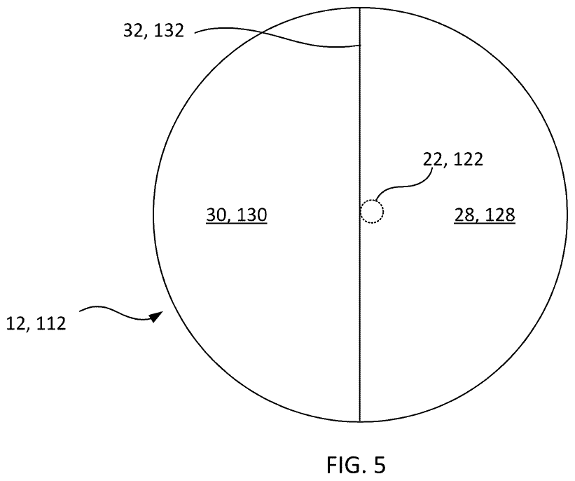

FIG. 5 illustrates a top view of the culture device 10 of FIGS. 1 to 4. As illustrated, there is provided a base 12, 112 with the first culture surface 28, 128 and the second culture surface 30, 130 separated by the adjoining region 32, 132. The optional outlet port 22 (present in FIGS. 1 and 2) is shown in dotted lines to illustrate the optional nature thereof. The optional outlet port 22 may be located on either culture surface 28, 128, 30, 130. Equally, there may be multiple outlet ports (not shown).

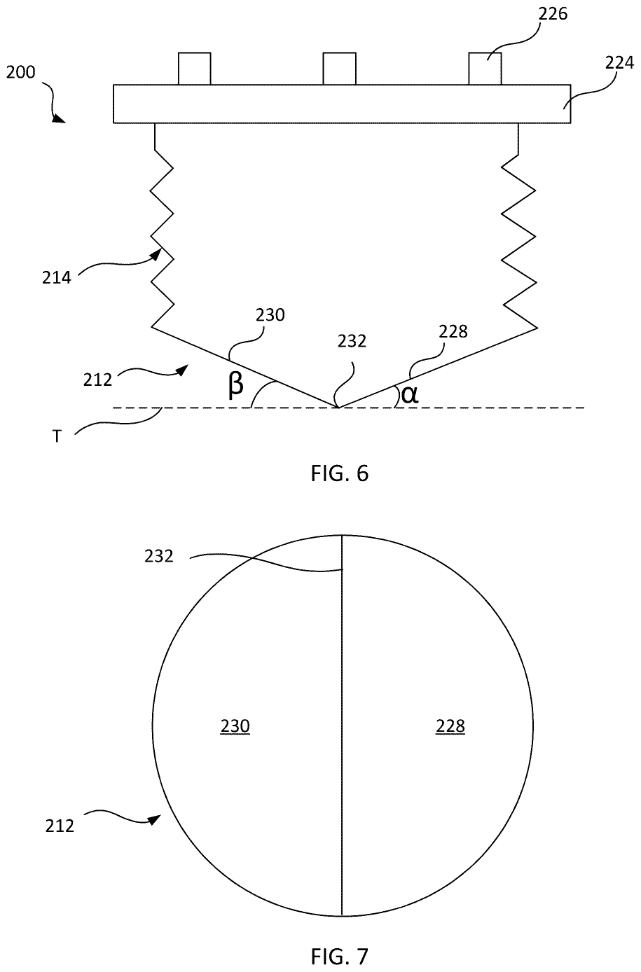

FIG. 6 illustrates a side view of another culture device 200. The culture device 200 is substantially similar to that described in FIGS. 3 and 4. In particular, the culture device 200 includes a base 212, a flexible side wall 214, an interface plate 224 and optional ports 226 as described above.

The culture device 200 includes a first culture surface 228 separated from a second culture surface 230 by an adjoining region 232. The first culture surface 228 and the second culture surface 230 are located at a fixed angled with respect to one another as will be described further herein. In this example, the first culture surface 228 forms a fixed angle α with respect to the transverse plane T, and the second culture surface 230 forms a fixed angle β with respect to the transverse plane T. The transverse plane T is partially defined by the base 212 in that the adjoining region 232 (i.e., an axis defined by the adjoining region 232) is disposed within the transverse plane T. The angles α and β are illustrated as being different in the present example. In other examples, they may be identical. In this particular example, α is 30 degrees and β is 45 degrees.

FIG. 7 illustrates the culture device 200 of FIG. 6 in a top view. As illustrated, the first culture surface 228 and the second culture surface 230 of the base 212 are separated by an adjoining region 232.

It is noted that whilst the culture device of FIGS. 6 and 7 is substantially similar to culture device 100 of FIGS. 3 and 4, the further features of the culture device 10 of FIGS. 1 and 2 may also be imported. For example, first culture surface 228 and/or second culture surface 230, or indeed the adjoining region 232, may include an optional outlet port. Further, the base 212 may be provided with a block of material disposed either side of the adjoining region 232 to provide the angled first and second culture surfaces 228, 230 whilst ensuring a continuous and planar second, i.e., underside, of the base 212.

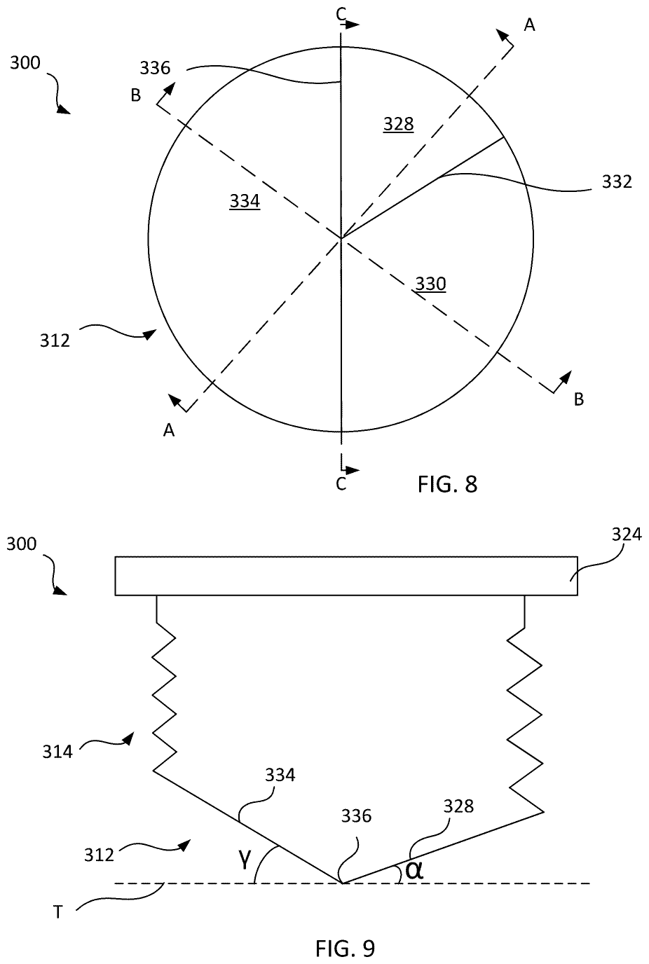

FIGS. 8 to 11 illustrate yet another culture device 300. The culture device 300 is substantially similar to the culture device 200 as described in relation to FIGS. 6 and 7, except in that it has an additional (i.e., three in total) culture surfaces. For example, as shown in FIGS. 9 to 11, the culture device 300 includes the features of the base 312, the flexible side wall 314, the interface plate 324 and the ports 326.

In particular, as shown in FIG. 8, the culture device 300 includes a base 312 that has a first culture surface 328, a second culture surface 330 and a third culture surface 334. The first culture surface 328, the second culture surface 330 and the third culture surface 334 are each located at a fixed angled with respect to one another as will be described further herein. The first culture surface 328 is separated from the second culture surface 330 by a first adjoining region 332. The second culture surface 330 is separated from the third culture surface 334 by a second adjoining region 336. The third culture surface 334 is separated from the first culture surface 328 by the second adjoining region 336. The first, second and third culture surface 328, 330, 334 are each formed as a sector, namely a circular sector where base 312 is circular as per this example. That is, each of the first, second and third culture surfaces 328, 330, 334 are formed by two radii and an arc.

FIG. 9 illustrates a sectional view through line A-A of FIG. 8, and illustrates the relationship between the first culture surface 328 and the third culture surface 334 separated by the second adjoining region 336. As shown, the first culture surface 328 forms a fixed angle α with respect to the transverse plane T. In this example, α is 20 degrees. As shown, the third culture surface 334 forms a fixed angle γ with respect to the transverse plane T. In this example, γ is 45 degrees.

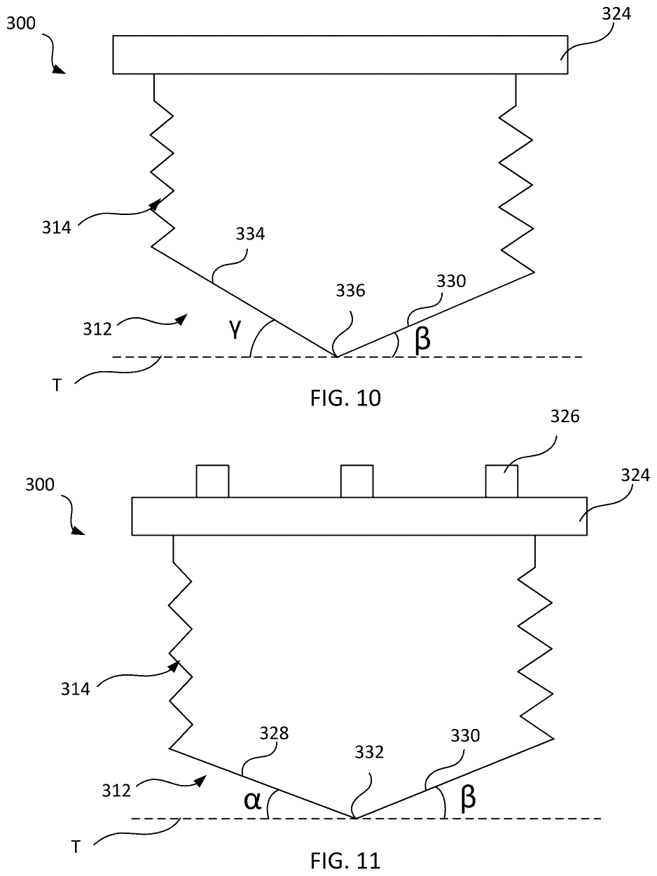

FIG. 10 illustrates a sectional view through line B-B of FIG. 8, and illustrates the relationship between the second culture surface 330 and the third culture surface 334 separated by the second adjoining region 336. As shown, the second culture surface 330 forms a fixed angle β with respect to the transverse plane T. In this example, β is 30 degrees. As shown, and also described above in relation to FIG. 9, the third culture surface 334 forms a fixed angle γ with respect to the transverse plane T. In the example shown, γ is 45 degrees.

FIG. 11 illustrates a sectional view through line C-C of FIG. 8, i.e., along the second adjoining region 336, and illustrates the relationship between the first culture surface 328 and the second culture surface 330 separated by the second adjoining region 336. As shown, and also described above in relation to FIG. 9, the first culture surface 328 forms a fixed angle α with respect to the transverse plane T. In the example shown, α is 20 degrees. As also shown and described above in relation to FIG. 10, the second culture surface 330 forms a fixed angle β with respect to the transverse plane T. In the example shown, β is 30 degrees.

As described above, the base 312 of the culture device 300 is thus formed of three angled, or slanted, cylindrical sectors thereby forming each of the culture surfaces 328, 330, 334. The surface area of each culture surface 328, 330, 334 is increasingly larger, from first culture surface 328 to third culture surface 334, and forms a ratio between the surface areas of 1:3:6 (i.e., surface areas of first culture surface: second culture surface: third culture surface). In the particular example, the first culture surface 328 has a surface area of 25 cm², the second culture surface 330 has a surface area of 75 cm², and the third culture surface 334 has a surface area of 150 cm². The “surface area” referred to is the surface that forms the respective fixed angle α, β, γ with respect to the transverse plane T. The area defining the “surface area,” i.e., the area of the culture surface that extends within a plane of the culture surface, defines an area upon which cells, tissues or other materials can culture.

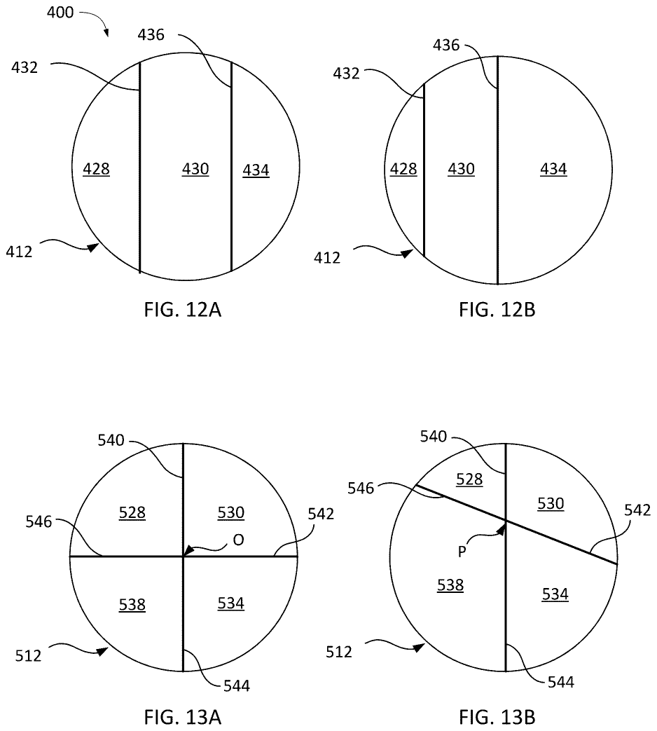

FIGS. 12A and 12B illustrate another culture device 400 having a base 412 including three culture surfaces: a first culture surface 428, a second culture surface 430, and a third culture surface 434. The first culture surface 428 is separated from the second culture surface 430 by a first adjoining region 432. The second culture surface 430 is separated from the third culture surface 434 by a second adjoining region 436. As shown in FIG. 12A, the first adjoining region 432 and the second adjoining region 436 each extend across a chord of the generally circular base 412, and are parallel with respect to one another. The first, second and third culture surfaces 428, 430, 434 each have an approximately equal surface area, such that the ratio between the first, second and third culture surfaces 428, 430, 434 is approximately 1:1:1. As shown in FIG. 12B, in another example, the first adjoining region 432 extends across a chord of the generally circular base 412, and the second adjoining region 436 extends across a diameter of the generally circular base 412. Thus, the first adjoining region 432 and the second adjoining region 436 extend parallel with respect to one another. The first, second and third culture surfaces 428, 430, 434 each have an increasingly larger surface area, such that the ratio between the first, second and third culture surfaces 428, 430, 434 is approximately 1:2:3 or, in another example, 1:3:6.

In either example, at least two of the culture surfaces 428, 430, 434 forms a fixed angle with respect to a transverse plane, analogous to that described in the previous examples. In a particular example, the first culture surface 428 and the third culture surface 434 form a fixed angle with respect to the transverse plane, whilst the second culture surface 430 is substantially parallel to, or co-planar with, the transverse plane. The first and third culture surface 428, 434 may be at equal, or different, fixed angles with respect to the transverse plane.

FIGS. 13A and 13B illustrate a culture device 500 having a base 512 including four culture surfaces: a first culture surface 528, a second culture surface 530, a third culture surface 534, and a fourth culture surface 538. There are also provided four adjoining regions: a first adjoining region 540, a second adjoining region 542, a third adjoining region 544 and a fourth adjoining region 546. The first culture surface 528 is separated from the second culture surface 530 by the first adjoining region 540. The second culture surface 530 is separated from the third culture surface 534 by a second adjoining region 542. The third culture surface 534 is separated from the fourth culture surface 538 by the third adjoining region 544. The fourth culture surface 538 is separated from the first culture surface 528 by the fourth adjoining region 546.

The difference between FIGS. 13A and 13B is the point at which the adjoining regions 540, 542, 544, 546 meet. In FIG. 13A, each adjoining region 540, 542, 544, 546 intersects at a central origin O, a point disposed along the central longitudinal axis of the device. In the example of FIG. 13A, the surface area of each culture surface 528, 530, 534, 538 is equal. Alternatively, each adjoining region 540, 542, 544, 546 intersects at a non-central point P, a point disposed along an axis other than the central longitudinal axis of the device. In the example of FIG. 13B, the surface area of each culture surface 528, 530, 534, 538 is different.

In either example, at least three of the culture surfaces 528, 530, 534, 538 forms a fixed angle with respect to a transverse plane, analogous to that described in the previous examples. In a particular example, the first culture surface 528, the second culture surface 530 and the third culture surface 534 each form a fixed angle with respect to the transverse plane, whilst the fourth culture surface 538 is substantially parallel to, or co-planar with, the transverse plane. The first, second and third culture surfaces 528, 530, 534 may be at equal, or different, fixed angles with respect to the transverse plane.

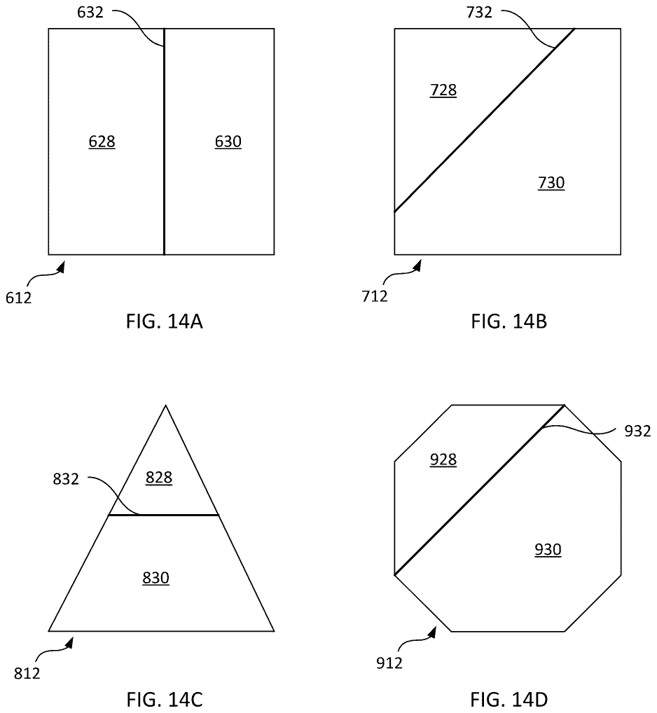

FIGS. 14A-14D illustrate different cross-sectional shapes of the base 612, 712, 812, 912 in a culture device, each having an adjoining region 632, 732, 832, 932 separating a first culture surface 628, 728, 828, 928 from a second culture surface 630, 730, 830, 930. FIG. 14A illustrates a generally square base 612 including an adjoining region 632 extending between opposing sides of the square base 612. FIG. 14B illustrates a generally square base 712 including an adjoining region 732 extending between adjacent sides of the square base 712. FIG. 14C illustrates a generally triangular base 812 including an adjoining region 832 extending between adjacent sides of the triangular base 812. FIG. 14D illustrates a generally octagonal base 912 including an adjoining region 932 extending between generally opposing corners of the octagonal base 912. As will be recognized by those skilled in the art, the base 912 may assume any geometry or cross-sectional profile, and the adjoining region 932 may extend between any appropriate edges, sides, corners or the like of such base 912.

In the examples of FIGS. 14A-14D, at least one, or both, of the first culture surface 628, 728, 828, 928 and the second culture surface 630, 730, 830, 930 forms a fixed angle with respect to the transverse plane T as described herein. The angles formed may be any of the angles contemplated herein.

The presently described culture devices of FIGS. 1 to 14D may be used to passage cells in the manner as described below. Particularly, the fixed nature of the angles between the culture surfaces (or, indeed between a certain culture surface and the traverse plane T), enable passaging of cells between the surfaces by moving the base (i.e., moving the traverse plane T at a predetermined angle) so that each culture surface is positioned—in a predetermined sequence—in a horizontal plane. In the particular exemplary protocols discussed below, reference is made to the use of the culture device 100 of FIGS. 3 and 4, although such use is equally applicable to any other culture device described herein.

### EXAMPLE 1

### Suspension-Type Cell Culture

In one example, a suspension-type cell culture, i.e., a suspension of cells of a desired type within an appropriate medium, is introduced into the cell culture device 100 shown in FIGS. 3 and 4. In particular, a cell culture may be loaded into the volume of the culture device 100 from an external receptacle having a needle-based connector (not shown). The needle-based connector may cause piercing of a septum seal within the external receptacle, and also may cause piercing of one of the ports 126 of the interface plate 124, thereby fluidly connecting the external receptacle and the culture device 100. Accordingly, the cell culture is introduced into the culture device 100.

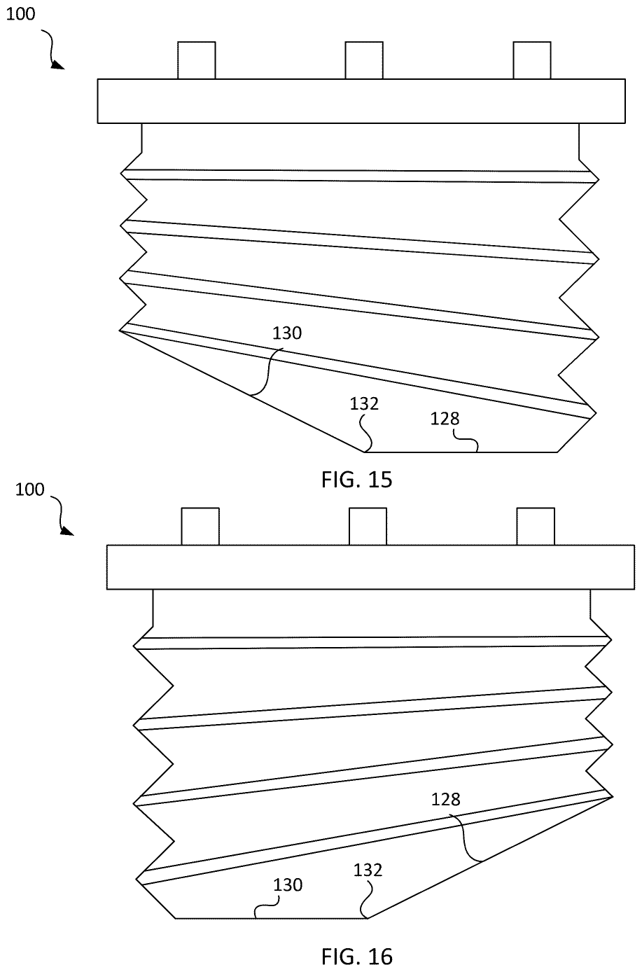

Either prior to introducing a suspension-type cell culture, or after the introduction thereof, an external actuator (not shown) may rotate the entirety of the base 112 such that the first culture surface 128 is substantially horizontal, i.e., planar to or co-planar with the transverse plane T. This is illustrated in FIG. 15. The external actuator (not shown) may pivot, or rotate, the base about an axis substantially aligned with the adjoining region 132. It is noted that the flexible side wall 114 allows for such pivoting of the base 112 into the shown in FIG. 15.

The cells within the cell culture are allowed to settle onto the base wall 112, namely on the first culture surfaces 128. Optionally, the base 112 may be agitated by the external actuated, e.g., pivoting about an axis defined by the adjoining region 132, so that all cells are moved to, and thus reside on, the first culture surface 128. Alternatively, the culture device 100 may be titled in its entirety so that cells settle and reside on the first culture surface 128.

The cells in culture are allowed to proliferate, such that exponential growth of cells is achieved. During proliferation, appropriate materials, such as growth factors, fresh medium, proteins, magnetic beads, antibodies, viruses or the like, may be added to the cell culture. Such materials may be introduced from an external receptacle having a needle-based connector analogously to the introduction of the cell culture discussed above. During proliferation, the base 112, i.e., the first culture surface 128 and the second culture surface 130, may be pivoted about an axis defined by the adjoining region 132 in a cyclical manner for the purposes of mixing and/or resuspending the cell culture. Generally, cyclical pivoting about an axis defined by the adjoining region 132 may induce turbulence within the cell culture device 100 to provide mixing and/or resuspension of the contents therein. The cyclical pivoting about an axis defined by the adjoining region 132 may be provided between any appropriate angle, such as +10 degrees and −10 degrees. The cell culture is allowed to proliferate until a desired cell density is achieved.

Optionally, a medium exchange step is provided. In particular, a portion of the cell culture medium, i.e., medium that is exhausted or spent, is removed from the culture device 100 and replaced with fresh medium. The medium may be removed through the port 126 of the interface plate 124. Fresh medium is introduced through the port 126 of the interface plate 124, through the use of an external receptacle having a needle-based connector, analogous to the introduction of the cell culture discussed above. Equally, as would be recognized by those skilled in the art, a medium exchange step may be provided after the passaging of the cells as discussed below. In some examples, a medium exchange step may be provided both prior to, and after, the passaging of the cells as discussed below.

The culture device 100, specifically the base 112 thereof, is pivoted about an axis defined by the adjoining region 132. In particular, the external actuator (not shown) causes pivoting of the entirety of the base 112 such that the second culture surface 130 is substantially horizontal, i.e., planar to or co-planar with the transverse plane T. This is illustrated in FIG. 16. In this way, the cells are transferred, or passaged, from the first culture surface 128 to the second culture surface 130, by virtue of gravity, so that all of the cells reside on the second culture surface 130. The cell culture may then be allowed to proliferate until a desired cell density is achieved. During proliferation, the base 112 may be pivoted about an axis defined by the adjoining region 132 in a cyclical manner for the purposes of mixing and/or resuspending the cell culture. Generally, cyclical pivoting about an axis defined by the adjoining region 132 may induce turbulence within the cell culture device 100 to provide mixing and/or resuspension of the contents therein. The cyclical pivoting about an axis defined the adjoining region 132 may be provided between an angle of +10 degrees and −10 degrees.

Optionally, further transferring, or passaging, of the cells may take place, namely from the second culture surface 130 back to the first culture surface 128. In this case, the above process is followed in reverse, i.e., the entirety of the base 112 is moved from the position of FIG. 16 back to the position of FIG. 15. In this way, a further mixing or passaging action is achieved, which may promote oxygenation of the cell culture. The cell culture is allowed to proliferate until a desired cell density is achieved.

Yet further, the pivoting of the base 112 about an axis defined by the adjoining region 132 may carried out for the purposes of mixing and/or resuspending the cell culture. Accordingly, following mixing and/or resuspending, the cells may be allowed to settle on one of the first and second culture surfaces 128, 130 depending on which culture surface is placed in the horizontal plane.

Finally, once the cell culture has reached maturity and the desired density has been achieved, the cell culture is harvested through a port 126 of the interface plate 124. Alternatively, in the case of a culture device with an optional port in the base thereof, cells may be harvested through such port.

### EXAMPLE 2

### Adherent-Type Cell Culture

In another example, an adherent-type cell culture, i.e., adherent-type cells within an appropriate medium, is introduced into the cell culture device 100 shown in FIGS. 3 and 4. The cell culture device 100 includes a coating or substrate on each of the first and second culture surfaces 128, 130 to allow for cell adhesion thereto. In particular, the cell culture may be loaded into the volume of the culture device 100 from an external receptacle having a needle-based connector (not shown). The needle-based connector may cause piercing of a septum seal within the external receptacle, and also may cause piercing of one of the ports 126 of the interface plate 124, thereby fluidly connecting the external receptacle and the culture device 100.

Either prior to introducing an adherent-type cell culture, or after the introduction thereof, an external actuator (not shown) may rotate the entirety of the base 112 such that the first culture surface 128 is substantially horizontal, i.e., planar to or co-planar with the transverse plane T. This is illustrated in FIG. 15. The external actuator (not shown) may pivot, or rotate, the base about an axis substantially aligned with the adjoining region 132. It is noted that the flexible side wall 114 allows for such pivoting of the base 112 into the shown in FIG. 15.

The cells within the cell culture are allowed to settle onto the base wall 112, namely on the first culture surface 128, particularly on the coating or substrate thereof.

The cell culture is allowed to proliferate, namely through adherence to the first culture surface 128, such that exponential growth of cells is achieved. The cells may be allowed to adhere to, and proliferate on, the first culture surface 128 for a specified period, for example, for a period of 10 minutes to 24 hours, and preferably for a period of 6 to 12 hours. During proliferation, appropriate materials, such as growth factors, fresh medium, proteins, magnetic beads, antibodies, viruses or the like, may be added to the cell culture. Such materials may be introduced from an external receptacle having a needle-based connector analogously to the introduction of the cell culture discussed above. The cell culture is allowed to proliferate until a desired cell density is achieved.

Optionally, a medium exchange step is provided prior to passaging of the cells from first culture surface 128 to second culture surface 130. In particular, a portion of the cell culture medium, i.e., medium that is exhausted or spent, is removed from the culture device 100 and replaced with fresh medium. The medium may be removed through the port 126 of the interface plate 124. Fresh medium is introduced through the port 126 of the interface plate 124, through the use of an external receptacle having a needle-based connector, analogous to the introduction of the cell culture discussed above. Equally, as would be recognized by those skilled in the art, a medium exchange step may be provided after the passaging of the cells as discussed below. In some examples, a medium exchange step may be provided both prior to, and after, the passaging of the cells as discussed below.

Prior to the transferring, or passaging, of the adherent-type cells from the first culture surface 128 to the second culture surface 130, the cell culture is washed. In particular, all of, or a portion of, the cell culture medium is removed from the culture device 100, through one of the ports 126 of the interface plate 124. Subsequently, a wash solution is introduced into the culture device 100. The wash solution may be a buffered saline solution, for example, phosphate buffered saline. Generally, any suitable wash solution may be used to ensure the removal of any agents, such as serum, calcium or magnesium, that would inhibit the action of the dissociation reagent discussed below. The culture device 100 is gently agitated, for example, through rocking, in order to wash the cell culture. The agitation may be achieved through means of moving the base 112 by an external actuator (not shown). The wash solution is then removed from the culture device 100, through one of the ports 126 of the interface plate 124.

Following washing, and prior to passaging of the adherent-type cells, a dissociation reagent is added to the culture device 100. The dissociation reagent may be pre-warmed to a temperature of 37 degrees Celsius prior to addition. The dissociation reagent may be trypsin or TrypLE™, or another reagent to provide dissociation of adherent-type cells from the coating or substrate on the first culture surface 128. The culture device 10 is gently agitated, for example, through rocking, to ensure complete dissociation of the cells from the first culture surface 128. The agitation may again be achieved by moving the base 112 by an external actuator (not shown). The culture device 100 may also be incubated at room temperature for a period of time.

Upon detachment of the adherent-type cells from the first culture surface 128, the base 112 is pivoted about an axis defined by the adjoining region 132. In particular, the external actuator (not shown) causes pivoting of the entirety of the base 112 such that the second culture surface 130 is substantially horizontal, i.e., planar to or co-planar with the transverse plane T. This is illustrated in FIG. 16. In this way, the cells are transferred, or passaged, from the first culture surface 128 to the second culture surface 130, by virtue of gravity, so that all of the cells reside on the second culture surface 130. Fresh cell culture medium may also be added to the culture device through one of the ports 126 in the interface plate 124 at this stage. The adherent-type cells are then allowed to adhere to the coating or substrate on the second culture surface 130. The cells are allowed to adhere and proliferate for a specified period, for example, for a period of 10 minutes to 24 hours, and preferably for a period of 6 to 12 hours. Once the cells have been allowed to adhere for the specified period of time, the cell culture medium containing the dissociation reagent is removed through one of the ports 126 of the interface plate 124. Fresh cell culture medium is then introduced through the port 126 of the interface plate 124, through the use of an external receptacle having a needle-based connector, analogous to the introduction of the cell culture discussed above, and the cells are allowed to proliferate on the coating or substrate on the second culture surface 130. During proliferation, appropriate materials, such as growth factors, fresh medium, proteins, magnetic beads, antibodies, viruses or the like, may be added to the cell culture. Such materials may be introduced from an external receptacle having a needle-based connector analogously to the introduction of the cell culture discussed above. The cell culture is allowed to proliferate until a desired cell density is achieved.

Finally, once the cell culture has reached maturity and the desired density has been achieved, the cell culture is harvested through a port 122 of the interface plate 124. Alternatively, in the case of a culture device with an optional port in the base thereof, cells may be harvested through such port. In particular, the washing and dissociation steps discussed above may be applied to the cell culture residing on the coating or substrate on the second culture surface 130, thereby ensuring appropriate dissociation of the adherent-type cells from the same. Subsequently, the base 112 may be moved or pivoted, by an actuator (not shown), about an axis defined by the adjoining region 132 to ensure all of the cells are detached from the coating or substrate. The cells may then be harvested in the manner described above.

Although the above example protocols describe two culture surfaces, the skilled person would equally apply the above teachings to any number of culture surfaces. In particular, in the case of three, four, or more culture surfaces, the base may likewise be moved so that cells are passaged from a first culture surface, to a second culture surface, to a third surface, and so on. For adherent-type cells, dissociation steps as described above are required between each passage.

Moreover, the above example protocols may be carried out in any device described herein. Yet further, the example protocols may be followed in any order, for example, suspension-or adherent-type cells may be cultured firstly on the second culture surface 130, followed by passaging to the first culture surface 128.

Moreover, whilst the use of the device noted above is described in respect of a movement of the base to allow transferring-or “passaging”-of material, it is equally contemplated that the device in its entirety may be moved, tilted or the like to allow for sequential surfaces to be positioned in a horizontal manner. This is particularly the case for examples in which the side wall is rigid.

It will be appreciated by persons skilled in the art that the above embodiments have been described by way of example only and not in any limitative sense, and that various alterations and modifications are possible without departing from the scope of the invention as defined by the appended claims. Various modifications to the detailed designs as described above are possible, for example, variations may exist in shape, size, material, arrangement, assembly or the like.

### In Certain Implementations, the Invention May Be Defined by One or More of the following numbered clauses:

Embodiment 1. A device (10, 100, 200, 300) comprising:
a base (12, 112, 212, 312, 412, 512, 612, 712, 812, 912) comprising a first surface (28, 128, 228, 328, 428, 528, 628, 728, 828, 928) and a second surface (30, 130, 230, 330, 430, 530, 630, 730, 830, 930) fixedly arranged with respect to the first surface; and
a side wall (14, 114, 214, 314) extending from the base and defining an internal volume of the device,
wherein the first surface and the second surface are non-coplanar.

Embodiment 2. The device according to embodiment 1, wherein the side wall is a flexible side wall, such as a compressible side wall.

Embodiment 3. The device according to embodiment 1 or embodiment 2, wherein the first surface forms a first angle (a) with respect to a transverse plane (T) extending substantially perpendicularly to a central longitudinal axis (L) of the device.

Embodiment 4. The device according to embodiment 3, wherein the first angle is from approximately 1 degree to approximately 45 degrees, preferably from approximately 5 degrees to approximately 30 degrees.

Embodiment 5. The device according to any preceding embodiment, wherein the second surface forms a second angle (B) with respect to a transverse plane (T) extending substantially perpendicularly to a central longitudinal axis (L) of the device.

Embodiment 6. The device according to embodiment 5, wherein the second angle is from approximately 1 degree to approximately 45 degrees, preferably from approximately 5 degrees to approximately 30 degrees.

Embodiment 7. The device according to any preceding embodiment, wherein a ratio between a surface area of the first surface and a surface area of the second surface is from approximately 1:9 to approximately 1:1.

Embodiment 8. The device according to embodiment 7, wherein the ratio is approximately 1:3.

Embodiment 9. The device according to any preceding embodiment, wherein the base further comprises a third surface (334, 434, 534) fixedly arranged with respect to each of the first surface and the second surface, wherein the first surface, the second surface and the third surface are non-coplanar.

Embodiment 10. The culture device according to embodiment 9, wherein the third surface forms a third angle (γ) with respect to a transverse plane (T) extending substantially perpendicularly to a central longitudinal axis (L) of the device.

Embodiment 11. The culture device according to embodiment 10, wherein the third angle is from approximately 1 degree to approximately 45 degrees, preferably from approximately 5 degrees to approximately 30 degrees.

Embodiment 12. The device according to any one of embodiment 9 to 11, wherein a ratio between a surface area of the first surface, the second surface and the third surface is approximately 1:2:3 or approximately 1:3:6.

Embodiment 13. The device according to any preceding embodiment, wherein the base further comprises at least one port (22).

Embodiment 14. The device according to any preceding embodiment, wherein at least one of the surfaces comprises an adherent cell culture substrate or coating.

Embodiment 15. The device according to embodiment 14, wherein each surface comprises an adherent cell culture substrate or coating.

Embodiment 16. The device according to any one of embodiment 14 or 15,wherein the adherent cell culture substrate or coating is selected from the group comprising collagen, fibronectin, vitronectin, laminin, gelatine, poly-lysine, cellulose or a combination thereof.

Embodiment 17. The device according to any preceding embodiment, wherein the base and/or at least one of the surfaces comprises polystyrene, polycarbonate, low-density polyethylene, high-density polyethylene, silicone or a thermoplastic elastomer.

Embodiment 18. The device according to any preceding embodiment, wherein the device is a culture device, preferably a cell culture device.

Embodiment 19. A system comprising:
the device according to any one of embodiment 1 to 18; and
an actuator configured to engage at least a portion of the device and move the device between a first configuration, in which the first surface is horizontal, and a second configuration, in which the second surface is horizontal.

Embodiment 20. A method of culturing biological material, comprising the steps of:
providing a first surface (28, 128, 228, 328, 428, 528, 628, 728, 828, 928) and a second surface (30, 130, 230, 330, 430, 530, 630, 730, 830, 930) that is fixedly arranged with respect to the first surface, wherein the first surface and the second surface are non-coplanar;
orientating the first surface into a plane that is substantially horizontal;
culturing biological material on the first surface;
orientating the second surface into a plane that is substantially horizontal, thereby transferring biological material from the first surface to the second surface; and
culturing biological material on the second surface.

Embodiment 21. The method according to embodiment 20, wherein the biological material comprises tissue or cells.

Embodiment 22. The method according to embodiment 21, wherein the cells are adherent-type cells and each of the first surface and the second surface comprise an adherent cell culture substrate or coating.

Embodiment 23. Use of the device according to any one of embodiment 1 to 18, or the system of embodiment 19, in the culture of adherent-type cells.

Embodiment 24. Use of the device according to any one of embodiment 1 to 18, or the system of embodiment 19, in the culture of suspension-type cells.

Embodiment 25. A kit of parts, comprising:
a device according to any one of embodiment 1 to 18; and
an interfacing member configured to be coupled to a top of the device.

1. A culture device comprising:
a base comprising a first surface and a second surface fixedly arranged with respect to the first surface; and
a flexible side wall extending from the base and defining an internal volume of the device,
wherein the first surface and the second surface are non-coplanar.

2. The culture device according to claim 1, wherein the side wall is a compressible side wall.

3. The culture device according to claim 1 or claim 2, wherein the first surface forms a first angle with respect to a transverse plane extending substantially perpendicularly to a central longitudinal axis of the device.

4. The culture device according to claim 3, wherein the first angle is from approximately 1 degree to approximately 45 degrees, preferably from approximately 5 degrees to approximately 30 degrees.

5. The culture device according to claim 1, wherein the second surface forms a second angle with respect to a transverse plane extending substantially perpendicularly to a central longitudinal axis of the device.

6. The culture device according to claim 5, wherein the second angle is from approximately 1 degree to approximately 45 degrees, preferably from approximately 5 degrees to approximately 30 degrees.

7. The culture device according to claim 1, wherein a ratio between a surface area of the first surface and a surface area of the second surface is from approximately 1:9 to approximately 1:1.

8. The culture device according to claim 7, wherein the ratio is approximately 1:3.

9. The culture device according to claim 1, wherein the base further comprises a third surface fixedly arranged with respect to each of the first surface and the second surface, wherein the first surface, the second surface and the third surface are non-coplanar.

10. The culture device according to claim 9, wherein the third surface forms a third angle with respect to a transverse plane extending substantially perpendicularly to a central longitudinal axis of the device.

11. The culture device according to claim 10, wherein the third angle is from approximately 1 degree to approximately 45 degrees, preferably from approximately 5 degrees to approximately 30 degrees.

12. The culture device according to claim 9, wherein a ratio between a surface area of the first surface, the second surface and the third surface is approximately 1:2:3 or approximately 1:3:6.

13. The culture device according to claim 1, wherein the base further comprises at least one port.

14. The culture device according to claim 1, wherein at least one of the surfaces comprises an adherent cell culture substrate or coating.

15. The culture device according to claim 14, wherein each surface comprises an adherent cell culture substrate or coating.

16. The culture device according to claim 14, wherein the adherent cell culture substrate or coating is selected from the group comprising collagen, fibronectin, vitronectin, laminin, gelatine, poly-lysine, cellulose or a combination thereof.

17. The culture device according to claim 1, wherein the base and/or at least one of the surfaces comprises polystyrene, polycarbonate, low-density polyethylene, high-density polyethylene, silicone or a thermoplastic elastomer.

18. The culture device according to claim 1, wherein the culture device is a cell culture device.

19. A system comprising:
a culture device, the culture device comprising:
a base comprising a first surface and a second surface fixedly arranged with respect to the first surface; and
a flexible side wall extending from the base and defining an internal volume of the device,
wherein the first surface and the second surface are non-coplanar; and
an actuator configured to engage at least a portion of the device and move the device between a first configuration, in which the first surface is horizontal, and a second configuration, in which the second surface is horizontal.

20. A method of culturing biological material, the method comprising,
providing a first surface and a second surface that is fixedly arranged with respect to the first surface, wherein the first surface and the second surface are non-coplanar;
orientating the first surface into a plane that is substantially horizontal;
culturing biological material on the first surface;
orientating the second surface into a plane that is substantially horizontal, thereby transferring biological material from the first surface to the second surface; and
culturing biological material on the second surface.

21. The method according to claim 20, wherein the biological material comprises tissue or cells.

22. The method according to claim 21, wherein the cells are adherent-type cells and each of the first surface and the second surface comprise an adherent cell culture substrate or coating.

23. (canceled)

24. (cancelled)

25. The system according to claim 19, comprising
an interfacing member configured to be coupled to a top of the culture device.
